(19) United States
(12) Patent Application Publication
(10) Pub. No.: US 2024/0277696 A1
(43) Pub. Date: Aug. 22, 2024

(54) METHODS OF TREATING NERVE AGENT-INDUCED SEIZURES

(71) Applicant: Proniras Corporation, Seattle, WA (US)

(72) Inventor: Christopher Francis TOOMBS, Sammamish, WA (US)

(21) Appl. No.: 18/566,735

(22) PCT Filed: Jun. 9, 2022
(86) PCT No.: PCT/US2022/032783

§ 371 Date: Dec. 4, 2023

Related U.S. Application Data
(60) Provisional application No. 63/208,885, filed on Jun. 9, 2021.

(51) Int. Cl. A61K 31/4725; A61K 31/5513; A61P 25/08

(52) U.S. Cl. CPC A61K 31/4725; A61K 31/5513; A61P 25/08

(57) ABSTRACT

The present disclosure relates to methods of treating a disease (e.g., a seizure) in a subject in need thereof, comprising administering to the subject a therapeutically effective amount of tezampanel, a pharmaceutically acceptable salt thereof, or a prodrug thereof, alone or in combination with a therapeutically effective amount of a benzodiazepine, a pharmaceutically acceptable salt thereof, or a prodrug thereof.

### RELATED APPLICATION

This application claims the benefit of priority to U.S. Provisional Application No. 63/208,885, filed Jun. 9, 2021, the disclosure of which is incorporated herein by reference.

### GOVERNMENT SUPPORT

This invention was made with government support under HHSO100201800008 awarded by Biomedical Advanced Research and Development Authority. The government has certain rights in the invention.

### BACKGROUND

Derangement of normal cortical and subcortical function in the central nervous system (CNS) can be brought about through a variety of injuries (e.g., head trauma), pathologies (e.g., pediatric epilepsies), other physiologic stimuli (e.g., electrolyte imbalance), or an external chemical stimulus (e.g., nerve agent) which may induce a seizure state in a subject. Tezampanel is a competitive antagonist at the kainate and AMPA subtypes of glutamate receptors. There is thus a need for a novel use of tezampanel in treating seizure and potentially preventing more permanent brain injury resulting from induction of a seizure state from a variety of physical or pharmacologic stimuli.

### SUMMARY

In some aspects, the present disclosure provides a method of treating a disease (e.g., a seizure) in a subject in need thereof, comprising administering to the subject:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

In some aspects, the present disclosure provides a method of treating a disease (e.g., a seizure) resistant to a treatment with the benzodiazepine in a subject in need thereof, comprising administering to the subject a therapeutically effective amount of tezampanel, a pharmaceutically acceptable salt thereof, or a prodrug thereof.

In some aspects, the present disclosure provides a method of treating a disease (e.g., a seizure) resistant to a treatment with the tezampanel in a subject in need thereof, comprising administering to the subject a therapeutically effective amount of benzodiazepine, a pharmaceutically acceptable salt thereof, or a prodrug thereof.

In some aspects, the present disclosure provides a combination for treating a disease in a subject in need thereof, wherein the combination comprises:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

In some aspects, the present disclosure provides tezampanel, a pharmaceutically acceptable salt, or a prodrug thereof for use in treating a disease (e.g., a seizure) resistant to a treatment with benzodiazepine in a subject in need thereof.

In some aspects, the present disclosure provides benzodiazepine, a pharmaceutically acceptable salt, or a prodrug thereof for use in treating a disease (e.g., a seizure) resistant to a treatment with tezampanel in a subject in need thereof.

In some aspects, the present disclosure provides use of a combination in the manufacture of a medicament for treating a disease (e.g., a seizure) in a subject in need thereof, wherein the combination comprises:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

In some aspects, the present disclosure provides use of tezampanel, a pharmaceutically acceptable salt, or a prodrug thereof in the manufacture of a medicament for treating a disease (e.g., a seizure) resistant to a treatment with benzodiazepine in a subject in need thereof.

In some aspects, the present disclosure provides use of benzodiazepine, a pharmaceutically acceptable salt, or a prodrug thereof in the manufacture of a medicament for treating a disease (e.g., a seizure) resistant to a treatment with tezampanel in a subject in need thereof.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims.

### BRIEF DESCRIPTION OF DRAWINGS

FIG. 1 is a graph showing the Electroencephalogram (EEG) derived seizure severity measured by a 7-point Likert Scale in rodents with nerve agent induced seizures being successfully treated with tezampanel.

### DETAILED DESCRIPTION

In some aspects, the present disclosure provides a method of treating a disease (e.g., a seizure) in a subject in need thereof, comprising administering to the subject a therapeutically effective amount of tezampanel, a pharmaceutically acceptable salt thereof, or a prodrug thereof.

In some aspects, the present disclosure provides a method of treating a disease (e.g., a seizure) in a subject in need thereof, comprising administering to the subject:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

In some aspects, the present disclosure provides a method of treating a disease (e.g., a seizure) resistant to a treatment with the benzodiazepine in a subject in need thereof, comprising administering to the subject a therapeutically effective amount of tezampanel, a pharmaceutically acceptable salt thereof, or a prodrug thereof.

In some aspects, the present disclosure provides a method of treating a disease (e.g., a seizure) resistant to a treatment with the tezampanel in a subject in need thereof, comprising administering to the subject a therapeutically effective amount of benzodiazepine, a pharmaceutically acceptable salt thereof, or a prodrug thereof.

### Treated Subjects and Diseases

In some embodiments, the subject is an animal.

In some embodiments, the subject is a human.

In some embodiments, the disease is a seizure.

In some embodiments, the seizure is an agent-induced seizure (i.e., a seizure being induced by an inducing agent).

In some embodiments, the inducing agent is a nerve agent, i.e., an agent disrupting the mechanisms by which nerves transfer messages to organs.

In some embodiments, the inducing agent is an insecticide.

In some embodiments, the inducing agent (e.g., insecticide) is a carbamate.

In some embodiments, the inducing agent (e.g., carbamate) is aldicarb, carbofuran, carbaryl, ethienocarb, fenobucarb, oxamyl, or methomyl.

In some embodiments, the inducing agent (e.g., insecticide) is an organophosphate.

In some embodiments, the inducing agent (e.g., organophosphate) is parathion, malathion, methyl parathion, chlorpyrifos, diazinon, dichlorvos, phosmet, fenitrothion, tetrachlorvinfos, azimethaphos, azinphosmethyl, or terbufos.

In some embodiments, the inducing agent is a nerve agent, i.e., an agent disrupting the mechanisms by which nerves transfer messages to organs.

In some embodiments, the inducing agent (e.g., nerve agent) is an organophosphate.

In some embodiments, the inducing agent (e.g., nerve agent) is a G-series nerve agent.

In some embodiments, the inducing agent (e.g., G-series nerve agent) is tabun (GA), sarin (GB), soman (GD), or cyclosarin (GF).

In some embodiments, the inducing agent (e.g., nerve agent) is a V-series nerve agent.

In some embodiments, the inducing agent (e.g., V-series nerve agent) is VE, VG, VM, VP, VR, VS, or VX.

In some embodiments, the inducing agent (e.g., nerve agent) is a novichok class agent.

In some embodiments, the inducing agent (e.g., nerve agent) is a carbamate.

In some embodiments, the inducing agent (e.g., carbamate) is EA-2192, EA-3148, EA-3990, or EA-4056.

In some embodiments, the seizure is resistant to a treatment without tezampanel, the pharmaceutically acceptable salt thereof, or the prodrug thereof.

In some embodiments, the seizure is resistant to a treatment with the benzodiazepine, the pharmaceutically acceptable salt thereof, or the prodrug thereof and without tezampanel, the pharmaceutically acceptable salt thereof, or the prodrug thereof.

### Administration of Tezampanel

### Dosage of Tezampanel

In some embodiments, the therapeutically effective amount of tezampanel, the pharmaceutically acceptable salt thereof, or the prodrug thereof reduces the frequency, severity, or duration of the seizure.

In some embodiments, the therapeutically effective amount of tezampanel, the pharmaceutically acceptable salt thereof, or the prodrug thereof arrests the seizure.

In some embodiments, the therapeutically effective amount of tezampanel, the pharmaceutically acceptable salt thereof, or the prodrug thereof alleviates an adversing effect of tezampanel, the pharmaceutically acceptable salt thereof, or the prodrug thereof.

In some embodiments, the adversing effect is a sedating effect.

In some embodiments, the therapeutically effective amount of tezampanel, the pharmaceutically acceptable salt thereof, or the prodrug thereof is administered in an oral dosage form (e.g., a tablet).

In some embodiments, the prodrug of tezampanel is dasolampenel or a pharmaceutically acceptable salt thereof. In some embodiments, a therapeutically effective amount of dasolampanel, or the pharmaceutically acceptable salt thereof, is administered in an oral dosage form (e.g., a tablet).

In some embodiments, the tezampanel, the pharmaceutically acceptable salt thereof, or the prodrug thereof (e.g., dasolampanel) is administered at a dosage (e.g., a human dosage (a human oral dosage)) of about 2.5 mg/kg or lower, about 2.4 mg/kg or lower, about 2.3 mg/kg or lower, about 2.2 mg/kg or lower, about 2.1 mg/kg or lower, about 2.0 mg/kg or lower, about 1.9 mg/kg or lower, about 1.8 mg/kg or lower, about 1.7 mg/kg or lower, about 1.6 mg/kg or lower, about 1.5 mg/kg or lower, about 1.4 mg/kg or lower, about 1.3 mg/kg or lower, about 1.2 mg/kg or lower, about 1.1 mg/kg or lower, about 1.0 mg/kg or lower, about 0.9 mg/kg or lower, about 0.8 mg/kg or lower, about 0.7 mg/kg or lower, about 0.6 mg/kg or lower, about 0.5 mg/kg or lower, about 0.4 mg/kg or lower, about 0.3 mg/kg or lower, about 0.2 mg/kg or lower, about 0.1 mg/kg or lower, about 0.09 mg/kg or lower, about 0.08 mg/kg or lower, about 0.07 mg/kg or lower, about 0.06 mg/kg or lower, about 0.05 mg/kg or lower, about 0.04 mg/kg or lower, about 0.03 mg/kg or lower, about 0.02 mg/kg or lower, or about 0.01 mg/kg or lower.

In some embodiments, the tezampanel, the pharmaceutically acceptable salt thereof, or the prodrug thereof (e.g., dasolampanel) is administered at a dosage (e.g., a human dosage (a human oral dosage)) of about 0.01 mg/kg or higher, about 0.02 mg/kg or higher, about 0.03 mg/kg or higher, about 0.04 mg/kg or higher, about 0.05 mg/kg or higher, about 0.06 mg/kg or higher, about 0.07 mg/kg or higher, about 0.08 mg/kg or higher, about 0.09 mg/kg or higher, about 0.1 mg/kg or higher, about 0.2 mg/kg or higher, about 0.3 mg/kg or higher, about 0.4 mg/kg or higher, about 0.5 mg/kg or higher, about 0.6 mg/kg or higher, about 0.7 mg/kg or higher, about 0.8 mg/kg or higher, about 0.9 mg/kg or higher, about 1.0 mg/kg or higher, about 1.1 mg/kg or higher, about 1.2 mg/kg or higher, about 1.3 mg/kg or higher, about 1.4 mg/kg or higher, or about 1.5 mg/kg or higher.

In some embodiments, the tezampanel, the pharmaceutically acceptable salt thereof, or the prodrug thereof (e.g., dasolampanel) is administered at a dosage (e.g., a human dosage (a human oral dosage)) of about 1.2±1.1 mg/kg, about 1.2±1.0 mg/kg, about 1.2±0.9 mg/kg, about 1.2±0.8 mg/kg, about 1.2±0.7 mg/kg, about 1.2±0.6 mg/kg, about 1.2±0.5 mg/kg, about 1.2±0.4 mg/kg, about 1.2±0.3 mg/kg, about 1.2±0.2 mg/kg, about 1.2±0.1 mg/kg, about 1.2±0.09 mg/kg, about 1.2±0.08 mg/kg, about 1.2±0.07 mg/kg, about 1.2±0.06 mg/kg, about 1.2±0.05 mg/kg, about 1.2±0.04 mg/kg, about 1.2±0.03 mg/kg, about 1.2±0.02 mg/kg, or about 1.2±0.01 mg/kg (e.g., about 1.2 mg/kg).

In some embodiments, the tezampanel, the pharmaceutically acceptable salt thereof, or the prodrug thereof (e.g., dasolampanel) is administered at a dosage (e.g., a human dosage (a human oral dosage)) of about 0.8±0.7 mg/kg, about 0.8±0.6 mg/kg, about 0.8±0.5 mg/kg, about 0.8±0.4 mg/kg, about 0.8±0.3 mg/kg, about 0.8±0.2 mg/kg, about 0.8±0.1 mg/kg, about 0.8±0.09 mg/kg, about 0.8±0.08 mg/kg, about 0.8±0.07 mg/kg, about 0.8±0.06 mg/kg, about 0.8±0.05 mg/kg, about 0.8±0.04 mg/kg, about 0.8±0.03 mg/kg, about 0.8±0.02 mg/kg, or about 0.8±0.01 mg/kg (e.g., about 0.8 mg/kg).

In some embodiments, the tezampanel, the pharmaceutically acceptable salt thereof, or the prodrug thereof (e.g., dasolampanel) is administered at a dosage (e.g., a human dosage (a human oral dosage)) of about 0.4±0.3 mg/kg, about 0.4±0.2 mg/kg, about 0.4±0.1 mg/kg, about 0.4±0.09 mg/kg, about 0.4±0.08 mg/kg, about 0.4±0.07 mg/kg, about 0.4±0.06 mg/kg, about 0.4±0.05 mg/kg, about 0.4±0.04 mg/kg, about 0.4±0.03 mg/kg, about 0.4±0.02 mg/kg, or about 0.4±0.01 mg/kg (e.g., about 0.4 mg/kg).

In some embodiments, the tezampanel, the pharmaceutically acceptable salt thereof, or the prodrug thereof (e.g., dasolampanel) is administered at a dosage (e.g., a human dosage (a human oral dosage)) of about 0.16±0.15 mg/kg, about 0.16±0.14 mg/kg, about 0.16±0.13 mg/kg, about 0.16±0.12 mg/kg, about 0.16±0.11 mg/kg, about 0.16±0.10 mg/kg, about 0.16±0.09 mg/kg, about 0.16±0.08 mg/kg, about 0.16±0.07 mg/kg, about 0.16±0.06 mg/kg, about 0.16±0.05 mg/kg, about 0.16±0.04 mg/kg, about 0.16±0.03 mg/kg, about 0.16±0.02 mg/kg, or about 0.16±0.01 mg/kg (about 0.16 mg/kg).

In some embodiments, the tezampanel, the pharmaceutically acceptable salt thereof, or the prodrug thereof (e.g., dasolampanel) is administered at a dosage (e.g., a human dosage (a human oral dosage)) of about 110 mg or lower, about 105 mg or lower, about 100 mg or lower, about 95 mg or lower, about 90 mg or lower, about 85 mg or lower, about 80 mg or lower, about 75 mg or lower, about 70 mg or lower, about 65 mg or lower, about 60 mg or lower, about 55 mg or lower, about 50 mg or lower, about 45 mg or lower, about 40 mg or lower, about 35 mg or lower, about 30 mg or lower, about 25 mg or lower, about 20 mg or lower, about 15 mg or lower, about 10 mg or lower, about 9 mg or lower, about 8 mg or lower, about 7 mg or lower, about 6 mg or lower, about 5 mg or lower, about 4 mg or lower, about 3 mg or lower, about 2 mg or lower, or about 1 mg or lower.

In some embodiments, the tezampanel, the pharmaceutically acceptable salt thereof, or the prodrug thereof (e.g., dasolampanel) is administered at a dosage (e.g., a human dosage (a human oral dosage)) of about 1 mg or higher, about 2 mg or higher, about 3 mg or higher, about 4 mg or higher, about 5 mg or higher, about 6 mg or higher, about 7 mg or higher, about 8 mg or higher, about 9 mg or higher, about 10 mg or higher, about 15 mg or higher, about 20 mg or higher, about 25 mg or higher, about 30 mg or higher, about 35 mg or higher, about 40 mg or higher, about 45 mg or higher, about 50 mg or higher, about 55 mg or higher, about 60 mg or higher, about 65 mg or higher, about 70 mg or higher, about 75 mg or higher, about 80 mg or higher, about 85 mg or higher, about 90 mg or higher, about 95 mg or higher, or about 100 mg or higher.

In some embodiments, the therapeutically effective amount of tezampanel, the pharmaceutically acceptable salt thereof, or the prodrug thereof is administered in a parenteral dosage form (e.g., an intravenous, intramuscular, subcutaneous, or intradermal dosage form).

In some embodiments, the tezampanel, the pharmaceutically acceptable salt thereof, or the prodrug thereof is administered at a dosage (e.g., a human dosage (a human parenteral dosage)) about 0.8 mg/kg or lower, about 0.7 mg/kg or lower, about 0.6 mg/kg or lower, about 0.5 mg/kg or lower, about 0.4 mg/kg or lower, about 0.3 mg/kg or lower, about 0.2 mg/kg or lower, about 0.1 mg/kg or lower, about 0.09 mg/kg or lower, about 0.08 mg/kg or lower, about 0.07 mg/kg or lower, about 0.06 mg/kg or lower, about 0.05 mg/kg or lower, about 0.04 mg/kg or lower, about 0.03 mg/kg or lower, about 0.02 mg/kg or lower, or about 0.01 mg/kg or lower.

In some embodiments, the tezampanel, the pharmaceutically acceptable salt thereof, or the prodrug thereof is administered at a dosage (e.g., a human dosage (a human parenteral dosage)) of about 0.01 mg/kg or higher, about 0.02 mg/kg or higher, about 0.03 mg/kg or higher, about 0.04 mg/kg or higher, about 0.05 mg/kg or higher, about 0.06 mg/kg or higher, about 0.07 mg/kg or higher, about 0.08 mg/kg or higher, about 0.09 mg/kg or higher, about 0.1 mg/kg or higher, about 0.2 mg/kg or higher, about 0.3 mg/kg or higher, about 0.4 mg/kg or higher, about 0.5 mg/kg or higher, about 0.6 mg/kg or higher, or about 0.7 mg/kg or higher.

### Administration Frequency of Tezampanel

In some embodiments, the administration of the therapeutically effective amount of tezampanel, the pharmaceutically acceptable salt thereof, or the prodrug thereof is repeated one or more times during the treatment.

In some embodiments, the administration of the therapeutically effective amount of tezampanel, the pharmaceutically acceptable salt thereof, or the prodrug thereof is repeated once, twice, three times, four times, five times, six times, seven times, or eight times during the treatment.

In some embodiments, the administration of the therapeutically effective amount of tezampanel, the pharmaceutically acceptable salt thereof, or the prodrug thereof is repeated once during the treatment.

In some embodiments, the administration of the therapeutically effective amount of tezampanel, the pharmaceutically acceptable salt thereof, or the prodrug thereof is repeated twice during the treatment.

In some embodiments, the administration of the therapeutically effective amount of tezampanel, the pharmaceutically acceptable salt thereof, or the prodrug thereof is repeated three times during the treatment.

In some embodiments, the administration of the therapeutically effective amount of tezampanel, the pharmaceutically acceptable salt thereof, or the prodrug thereof is repeated every one hour, every two hours, every three hours, every four hours, every five hours, every six hours, every seven hours, every eight hours, every nine hours, every ten hours, every 11 hours, every 12 hours, every 13 hours, every 14 hours, every 15 hours, every 16 hours, every 17 hours, every 18 hours, every 19 hours, every 20 hours, every 21 hours, every 22 hours, every 23 hours, or every 24 hours.

In some embodiments, the administration of the therapeutically effective amount of tezampanel, the pharmaceutically acceptable salt thereof, or the prodrug thereof is repeated every four hours.

In some embodiments, the administration of the therapeutically effective amount of tezampanel, the pharmaceutically acceptable salt thereof, or the prodrug thereof is repeated every six hours.

In some embodiments, the administration of the therapeutically effective amount of tezampanel, the pharmaceutically acceptable salt thereof, or the prodrug thereof is repeated every eight hours.

### Benzodiazepines

The term “benzodiazepine”, as used here, refers to a compound with a core chemical structure being the fusion of a benzene ring and a diazepine ring.

In some embodiments, the benzodiazepine is clorazepate, diazepam, flurazepam, halazepam, prazepam, lorazepam, lormetazepam, oxazepam, temazepam, clonazepam, flunitrazepam, nimetazepam, nitrazepam, adinazolam, alprazolam, estazolam, triazolam, climazolam, loprazolam, or midazolam.

In some embodiments, the benzodiazepine is clorazepate, diazepam, flurazepam, halazepam, or prazepam.

In some embodiments, the benzodiazepine is lorazepam, lormetazepam, oxazepam, or temazepam.

In some embodiments, the benzodiazepine is clonazepam, flunitrazepam, nimetazepam, or nitrazepam.

In some embodiments, the benzodiazepine is adinazolam, alprazolam, estazolam, or triazolam.

In some embodiments, the benzodiazepine is climazolam, loprazolam, or midazolam.

### Dosage of Benzodiazepines

In some embodiments, the therapeutically effective amount of the benzodiazepine, the pharmaceutically acceptable salt thereof, or the prodrug thereof reduces the frequency, severity, or duration of the seizure.

In some embodiments, the therapeutically effective amount of the benzodiazepine, the pharmaceutically acceptable salt thereof, or the prodrug thereof arrests the seizure.

In some embodiments, the therapeutically effective amount of the benzodiazepine, the pharmaceutically acceptable salt thereof, or the prodrug thereof alleviates an adversing effect of the benzodiazepine, the pharmaceutically acceptable salt thereof, or the prodrug thereof.

In some embodiments, the adversing effect is a sedating effect.

In some embodiments, the therapeutically effective amount of the benzodiazepine, the pharmaceutically acceptable salt thereof, or the prodrug thereof is administered in an oral dosage form (e.g., a tablet).

In some embodiments, the therapeutically effective amount of the benzodiazepine, the pharmaceutically acceptable salt thereof, or the prodrug thereof is administered in a parenteral dosage form (e.g., an intravenous, intramuscular, subcutaneous, or intradermal dosage form).

### Administration Frequency of Benzodiazepine

In some embodiments, the administration of the therapeutically effective amount of the benzodiazepine, the pharmaceutically acceptable salt thereof, or the prodrug thereof is repeated one or more times during the treatment.

In some embodiments, the administration of the therapeutically effective amount of the benzodiazepine, the pharmaceutically acceptable salt thereof, or the prodrug thereof is repeated once, twice, three times, four times, five times, six times, seven times, or eight times during the treatment.

In some embodiments, the administration of the therapeutically effective amount of the benzodiazepine, the pharmaceutically acceptable salt thereof, or the prodrug thereof is repeated once during the treatment.

In some embodiments, the administration of the therapeutically effective amount of the benzodiazepine, the pharmaceutically acceptable salt thereof, or the prodrug thereof is repeated twice during the treatment.

In some embodiments, the administration of the therapeutically effective amount of the benzodiazepine, the pharmaceutically acceptable salt thereof, or the prodrug thereof is repeated three times during the treatment.

In some embodiments, the administration of the therapeutically effective amount of the benzodiazepine, the pharmaceutically acceptable salt thereof, or the prodrug thereof is repeated every one hour, every two hours, every three hours, every four hours, every five hours, every six hours, every seven hours, every eight hours, every nine hours, every ten hours, every 11 hours, every 12 hours, every 13 hours, every 14 hours, every 15 hours, every 16 hours, every 17 hours, every 18 hours, every 19 hours, every 20 hours, every 21 hours, every 22 hours, every 23 hours, or every 24 hours.

In some embodiments, the administration of the therapeutically effective amount of the benzodiazepine, the pharmaceutically acceptable salt thereof, or the prodrug thereof is repeated every four hours.

In some embodiments, the administration of the therapeutically effective amount of the benzodiazepine, the pharmaceutically acceptable salt thereof, or the prodrug thereof is repeated every six hours.

In some embodiments, the administration of the therapeutically effective amount of the benzodiazepine, the pharmaceutically acceptable salt thereof, or the prodrug thereof is repeated every eight hours.

Exemplary Relationship between Tezampanel and Benzodiazepine Administrations

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered simultaneously.

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered sequentially.

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered alternatively.

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in temporal proximity.

In some embodiments, the therapeutically effective amount of tezampanel, the pharmaceutically acceptable salt thereof, or the prodrug thereof is administered within about 1 minutes, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 18 hours, or about 24 hours after the administration of the therapeutically effective amount of the benzodiazepine, the pharmaceutically acceptable salt thereof.

In some embodiments, the therapeutically effective amount of the benzodiazepine, the pharmaceutically acceptable salt thereof, or the prodrug thereof is administered within about 1 minutes, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 18 hours, or about 24 hours after the administration of the therapeutically effective amount of tezampanel, the pharmaceutically acceptable salt thereof.

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in different administration routes.

In some embodiments, (i) the therapeutically effective amount of tezampanel, the pharmaceutically acceptable salt thereof, or the prodrug thereof (e.g., dasolampanel) and (ii) the therapeutically effective amount of the benzodiazepine, the pharmaceutically acceptable salt thereof, or the prodrug thereof are administered in a same administration route (e.g., oral administration).

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in separate formulations.

In some embodiments, (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in a co-formulation.

### Other Aspects of the Methods

In some embodiments, the method further comprises administering to the subject a supportive care.

In some embodiments, the supportive care comprises an anticholinergic agent. In some embodiments, the supportive care comprises atropine.

In some embodiments, the supportive care comprises an oxime being capable of protecting the active site of acetylcholinesterase (e.g., by competing against the action of an organophosphate or carbamate). In some embodiments, the supportive care comprises pralidoxime.

In some embodiments, the supportive care comprises an anticonvulsant. In some embodiments, the supportive care comprises diazepam.

In some embodiments, the supportive care comprises oxygen supplementation (e.g., for treating shortness of breath or hypoxia).

In some embodiments, the supportive care comprises warmth, hydration, or a combination thereof.

In some embodiments, the supportive care comprises warmth. In some embodiments, the supportive care comprises a heating source (e.g., for maintaining body temperature). In some embodiments, the supportive care comprises a blanket.

In some embodiments, the supportive care comprises hydration.

In some embodiments, the supportive care comprises an intravenous fluid (e.g., for counteracting fluid loss due to vomiting or diarrhea).

### Effects of the Administration

In some embodiments, the administration reduces the frequency, severity, or duration of the seizure.

In some embodiments, the therapeutically effective amount of tezampanel, the pharmaceutically acceptable salt thereof, or the prodrug thereof arrests the seizure.

In some embodiments, the frequency, severity, and/or duration of the seizure in the subject is measured by a Likert Scale Score (LSS) of the described in Table 1 below.

TABLE 1

| Likert Scale | |
|---|---|
| Score (LSS) | Electroencephalogram (EEG) Characterization |
| 0 | Normal EEG relative to the animal's state (awake or asleep) with no sharp |
| | waves, spikes, slowing, or seizuires. |
| 1 | Sporadic, infrequency abnormalities (spikes, sharp waves, gamma bursts, |
| | etc.) not clearly abnormal; considered within the range of normal EEG. |
| 2 | Low incidence, intermittent EEG abnormalities with no severe paroxysms |
| | associated with convusions (e.g., less than 10 spikes per interval). |
| 3 | High incident of intermittent abnormalities with no severe paroxysms |
| | associated with convulsions (e.g., more thean 10 spikes, up to 50% or the |
| | interval). |
| 4 | Frequent spikes or short synchronous epileptiform discharges, or semi- |
| | continuous spike and wave SE with no severe paroxysms associated with |
| | convulsions (considered as nonconvulsive SE). |
| 5 | Continuous nonconvulsive spike and wave activity (low or moderately |
| | powered). Includes low amplitude variants. |
| 6 | Continuous, high powered nonconvulsive spike and wave activity. |
| 7 | Convulsive status epilepticus or the presence of at least one frank seizure |
| | associated with convulsions during the scored interval. |

In some embodiments, the administration results in a reduced LSS of the subject.

In some embodiments, the subject has a lower LLS (e.g., by about 1, about 2, about 3, about 4, about 5, or about 6), as compared to a comparable subject (e.g., without administration of tezampanel, the pharmaceutically acceptable salt thereof, or the prodrug thereof), at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, or about 48 hours after the administration of tezampanel, the pharmaceutically acceptable salt thereof, or the prodrug thereof.

In some embodiments, the subject has a LLS of about 6 or lower, about 5 or lower, about 4 or lower, about 3 or lower, or about 2 or lower (e.g., about 1), at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, or about 48 hours after the administration of tezampanel, the pharmaceutically acceptable salt thereof, or the prodrug thereof.

In some embodiments, the subject has a LLS of about 5 or lower, about 4 or lower, about 3 or lower, or about 2 or lower (e.g., about 1), at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, or about 48 hours after the administration of tezampanel, the pharmaceutically acceptable salt thereof, or the prodrug thereof.

In some embodiments, the subject has a LLS of about 4 or lower, about 3 or lower, or about 2 or lower (e.g., about 1), at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours after the administration of tezampanel, the pharmaceutically acceptable salt thereof, or the prodrug thereof.

In some embodiments, the subject has a LLS of about 3 or lower, or about 2 or lower (e.g., about 1), at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, or about 12 hours after the administration of tezampanel, the pharmaceutically acceptable salt thereof, or the prodrug thereof.

### Exemplary Embodiments

Exemplary Embodiment No. 1. A method of treating a disease in a subject in need thereof, comprising administering to the subject: (i) a therapeutically effective amount of tezampanel, a pharmaceutically acceptable salt thereof, or a prodrug thereof; and (ii) a therapeutically effective amount of a benzodiazepine, a pharmaceutically acceptable salt thereof, or a prodrug thereof.

Exemplary Embodiment No. 2. A method of treating a disease resistant to a treatment with the benzodiazepine in a subject in need thereof, comprising administering to the subject a therapeutically effective amount of tezampanel, a pharmaceutically acceptable salt thereof, or a prodrug thereof.

Exemplary Embodiment No. 3. A method of treating a disease resistant to a treatment with the tezampanel in a subject in need thereof, comprising administering to the subject a therapeutically effective amount of benzodiazepine, a pharmaceutically acceptable salt thereof, or a prodrug thereof.

Exemplary Embodiment No. 4. A combination for treating a disease in a subject in need thereof, wherein the combination comprises: (i) a therapeutically effective amount of tezampanel, a pharmaceutically acceptable salt thereof, or a prodrug thereof; and (ii) a therapeutically effective amount of a benzodiazepine, a pharmaceutically acceptable salt thereof, or a prodrug thereof.

Exemplary Embodiment No. 5. Tezampanel, a pharmaceutically acceptable salt, or a prodrug thereof for use in treating a disease resistant to a treatment with benzodiazepine in a subject in need thereof.

Exemplary Embodiment No. 6. Benzodiazepine, a pharmaceutically acceptable salt, or a prodrug thereof for use in treating a disease resistant to a treatment with tezampanel in a subject in need thereof.

Exemplary Embodiment No. 7. Use of a combination in the manufacture of a medicament for treating a disease in a subject in need thereof, wherein the combination comprises: (i) a therapeutically effective amount of tezampanel, a pharmaceutically acceptable salt thereof, or a prodrug thereof; and (ii) a therapeutically effective amount of a benzodiazepine, a pharmaceutically acceptable salt thereof, or a prodrug thereof.

Exemplary Embodiment No. 8. Use of tezampanel, a pharmaceutically acceptable salt, or a prodrug thereof in the manufacture of a medicament for treating a disease resistant to a treatment with benzodiazepine in a subject in need thereof.

Exemplary Embodiment No. 9. Use of benzodiazepine, a pharmaceutically acceptable salt, or a prodrug thereof in the manufacture of a medicament for treating a disease resistant to a treatment with tezampanel in a subject in need thereof.

Exemplary Embodiment No. 10. The method, combination, tezampanel, benzodiazepine, or use of any one of the previous Exemplary Embodiments, wherein the subject is an animal.

Exemplary Embodiment No. 11. The method, combination, tezampanel, benzodiazepine, or use of any one of the previous Exemplary Embodiments, wherein the subject is a human.

Exemplary Embodiment No. 12. The method, combination, tezampanel, benzodiazepine, or use of any one of the previous Exemplary Embodiments, wherein the disease is a seizure.

Exemplary Embodiment No. 13. The method, combination, tezampanel, benzodiazepine, or use of any one of the previous Exemplary Embodiments, wherein the seizure is an agent-induced seizure.

Exemplary Embodiment No. 14. The method, combination, tezampanel, benzodiazepine, or use of any one of the previous Exemplary Embodiments, wherein the inducing agent is a nerve agent.

Exemplary Embodiment No. 15. The method, combination, tezampanel, benzodiazepine, or use of any one of the previous Exemplary Embodiments, wherein the inducing agent is an insecticide.

Exemplary Embodiment No. 16. The method, combination, tezampanel, benzodiazepine, or use of any one of the previous Exemplary Embodiments, wherein the insecticide is a carbamate.

Exemplary Embodiment No. 17. The method, combination, tezampanel, benzodiazepine, or use of any one of the previous Exemplary Embodiments, wherein the carbamate is aldicarb, carbofuran, carbaryl, ethienocarb, fenobucarb, oxamyl, or methomyl.

Exemplary Embodiment No. 18. The method, combination, tezampanel, benzodiazepine, or use of any one of the previous Exemplary Embodiments, wherein the insecticide is an organophosphate.

Exemplary Embodiment No. 19. The method, combination, tezampanel, benzodiazepine, or use of any one of the previous Exemplary Embodiments, wherein the organophosphate is parathion, malathion, methyl parathion, chlorpyrifos, diazinon, dichlorvos, phosmet, fenitrothion, tetrachlorvinfos, azimethaphos, azinphosmethyl, or terbufos.

Exemplary Embodiment No. 20. The method, combination, tezampanel, benzodiazepine, or use of any one of the previous Exemplary Embodiments, wherein the inducing agent is a nerve agent.

Exemplary Embodiment No. 21. The method, combination, tezampanel, benzodiazepine, or use of any one of the previous Exemplary Embodiments, wherein the nerve agent is an organophosphate.

Exemplary Embodiment No. 22. The method, combination, tezampanel, benzodiazepine, or use of any one of the previous Exemplary Embodiments, wherein the nerve agent is a G-series nerve agent.

Exemplary Embodiment No. 23. The method, combination, tezampanel, benzodiazepine, or use of any one of the previous Exemplary Embodiments, wherein the G-series nerve agent is tabun (GA), sarin (GB), soman (GD), or cyclosarin (GF).

Exemplary Embodiment No. 24. The method, combination, tezampanel, benzodiazepine, or use of any one of the previous Exemplary Embodiments, wherein the nerve agent is a V-series nerve agent.

Exemplary Embodiment No. 25. The method, combination, tezampanel, benzodiazepine, or use of any one of the previous Exemplary Embodiments, wherein the V-series nerve agent is VE, VG, VM, VP, VR, VS, or VX.

Exemplary Embodiment No. 26. The method, combination, tezampanel, benzodiazepine, or use of any one of the previous Exemplary Embodiments, wherein the nerve agent is a novichok class agent.

Exemplary Embodiment No. 27. The method, combination, tezampanel, benzodiazepine, or use of any one of the previous Exemplary Embodiments, wherein the nerve agent is a carbamate.

Exemplary Embodiment No. 28. The method, combination, tezampanel, benzodiazepine, or use of any one of the previous Exemplary Embodiments, wherein the carbamate is EA-2192, EA-3148, EA-3990, or EA-4056.

Exemplary Embodiment No. 29. The method, combination, tezampanel, benzodiazepine, or use of any one of the previous Exemplary Embodiments, wherein the seizure is resistant to a treatment without tezampanel, the pharmaceutically acceptable salt thereof, or the prodrug thereof.

Exemplary Embodiment No. 30. The method, combination, tezampanel, benzodiazepine, or use of any one of the previous Exemplary Embodiments, wherein the seizure is resistant to a treatment with the benzodiazepine, the pharmaceutically acceptable salt thereof, or the prodrug thereof and without tezampanel, the pharmaceutically acceptable salt thereof, or the prodrug thereof.

Exemplary Embodiment No. 31. The method, combination, tezampanel, benzodiazepine, or use of any one of the previous Exemplary Embodiments, wherein the therapeutically effective amount of tezampanel, the pharmaceutically acceptable salt thereof, or the prodrug thereof reduces the frequency, severity, or duration of the seizure.

Exemplary Embodiment No. 32. The method, combination, tezampanel, benzodiazepine, or use of any one of the previous Exemplary Embodiments, wherein the therapeutically effective amount of tezampanel, the pharmaceutically acceptable salt thereof, or the prodrug thereof arrests the seizure.

Exemplary Embodiment No. 33. The method, combination, tezampanel, benzodiazepine, or use of any one of the previous Exemplary Embodiments, wherein the therapeutically effective amount of tezampanel, the pharmaceutically acceptable salt thereof, or the prodrug thereof alleviates an adversing effect of tezampanel, the pharmaceutically acceptable salt thereof, or the prodrug thereof.

Exemplary Embodiment No. 34. The method, combination, tezampanel, benzodiazepine, or use of any one of the previous Exemplary Embodiments, wherein the adversing effect is a sedating effect.

Exemplary Embodiment No. 35. The method, combination, tezampanel, benzodiazepine, or use of any one of the previous Exemplary Embodiments, wherein the therapeutically effective amount of tezampanel, the pharmaceutically acceptable salt thereof, or the prodrug thereof is administered in an oral dosage form.

Exemplary Embodiment No. 36. The method, combination, tezampanel, benzodiazepine, or use of any one of the previous Exemplary Embodiments, wherein the prodrug of tezampanel is dasolampenel or a pharmaceutically acceptable salt thereof.

Exemplary Embodiment No. 37. The method, combination, tezampanel, benzodiazepine, or use of any one of the previous Exemplary Embodiments, wherein a therapeutically effective amount of dasolampanel, or the pharmaceutically acceptable salt thereof, is administered in an oral dosage form.

Exemplary Embodiment No. 38. The method, combination, tezampanel, benzodiazepine, or use of any one of the previous Exemplary Embodiments, wherein the oral dosage form is a tablet.

Exemplary Embodiment No. 39. The method, combination, tezampanel, benzodiazepine, or use of any one of the previous Exemplary Embodiments, wherein the tezampanel, the pharmaceutically acceptable salt thereof, or the prodrug thereof is administered at a dosage of about 2.5 mg/kg or lower, about 2.4 mg/kg or lower, about 2.3 mg/kg or lower, about 2.2 mg/kg or lower, about 2.1 mg/kg or lower, about 2.0 mg/kg or lower, about 1.9 mg/kg or lower, about 1.8 mg/kg or lower, about 1.7 mg/kg or lower, about 1.6 mg/kg or lower, about 1.5 mg/kg or lower, about 1.4 mg/kg or lower, about 1.3 mg/kg or lower, about 1.2 mg/kg or lower, about 1.1 mg/kg or lower, about 1.0 mg/kg or lower, about 0.9 mg/kg or lower, about 0.8 mg/kg or lower, about 0.7 mg/kg or lower, about 0.6 mg/kg or lower, about 0.5 mg/kg or lower, about 0.4 mg/kg or lower, about 0.3 mg/kg or lower, about 0.2 mg/kg or lower, about 0.1 mg/kg or lower, about 0.09 mg/kg or lower, about 0.08 mg/kg or lower, about 0.07 mg/kg or lower, about 0.06 mg/kg or lower, about 0.05 mg/kg or lower, about 0.04 mg/kg or lower, about 0.03 mg/kg or lower, about 0.02 mg/kg or lower, or about 0.01 mg/kg or lower.

Exemplary Embodiment No. 40. The method, combination, tezampanel, benzodiazepine, or use of any one of the previous Exemplary Embodiments, wherein the tezampanel, the pharmaceutically acceptable salt thereof, or the prodrug thereof is administered at a dosage of about 0.01 mg/kg or higher, about 0.02 mg/kg or higher, about 0.03 mg/kg or higher, about 0.04 mg/kg or higher, about 0.05 mg/kg or higher, about 0.06 mg/kg or higher, about 0.07 mg/kg or higher, about 0.08 mg/kg or higher, about 0.09 mg/kg or higher, about 0.1 mg/kg or higher, about 0.2 mg/kg or higher, about 0.3 mg/kg or higher, about 0.4 mg/kg or higher, about 0.5 mg/kg or higher, about 0.6 mg/kg or higher, about 0.7 mg/kg or higher, about 0.8 mg/kg or higher, about 0.9 mg/kg or higher, about 1.0 mg/kg or higher, about 1.1 mg/kg or higher, about 1.2 mg/kg or higher, about 1.3 mg/kg or higher, about 1.4 mg/kg or higher, or about 1.5 mg/kg or higher.

Exemplary Embodiment No. 41. The method, combination, tezampanel, benzodiazepine, or use of any one of the previous Exemplary Embodiments, wherein the tezampanel, the pharmaceutically acceptable salt thereof, or the prodrug thereof is administered at a dosage of about 1.2±1.1 mg/kg, about 1.2±1.0 mg/kg, about 1.2±0.9 mg/kg, about 1.2±0.8 mg/kg, about 1.2±0.7 mg/kg, about 1.2±0.6 mg/kg, about 1.2±0.5 mg/kg, about 1.2±0.4 mg/kg, about 1.2±0.3 mg/kg, about 1.2±0.2 mg/kg, about 1.2±0.1 mg/kg, about 1.2±0.09 mg/kg, about 1.2±0.08 mg/kg, about 1.2±0.07 mg/kg, about 1.2±0.06 mg/kg, about 1.2±0.05 mg/kg, about 1.2±0.04 mg/kg, about 1.2±0.03 mg/kg, about 1.2±0.02 mg/kg, or about 1.2±0.01 mg/kg.

Exemplary Embodiment No. 42. The method, combination, tezampanel, benzodiazepine, or use of any one of the previous Exemplary Embodiments, wherein the tezampanel, the pharmaceutically acceptable salt thereof, or the prodrug thereof is administered at a dosage of about 0.8±0.7 mg/kg, about 0.8±0.6 mg/kg, about 0.8±0.5 mg/kg, about 0.8±0.4 mg/kg, about 0.8±0.3 mg/kg, about 0.8±0.2 mg/kg, about 0.8±0.1 mg/kg, about 0.8±0.09 mg/kg, about 0.8±0.08 mg/kg, about 0.8±0.07 mg/kg, about 0.8±0.06 mg/kg, about 0.8±0.05 mg/kg, about 0.8±0.04 mg/kg, about 0.8±0.03 mg/kg, about 0.8±0.02 mg/kg, or about 0.8±0.01 mg/kg.

Exemplary Embodiment No. 43. The method, combination, tezampanel, benzodiazepine, or use of any one of the previous Exemplary Embodiments, wherein the tezampanel, the pharmaceutically acceptable salt thereof, or the prodrug thereof is administered at a dosage of about 0.4±0.3 mg/kg, about 0.4±0.2 mg/kg, about 0.4±0.1 mg/kg, about 0.4±0.09 mg/kg, about 0.4±0.08 mg/kg, about 0.4±0.07 mg/kg, about 0.4±0.06 mg/kg, about 0.4±0.05 mg/kg, about 0.4±0.04 mg/kg, about 0.4±0.03 mg/kg, about 0.4±0.02 mg/kg, or about 0.4±0.01 mg/kg.

Exemplary Embodiment No. 44. The method, combination, tezampanel, benzodiazepine, or use of any one of the previous Exemplary Embodiments, wherein the tezampanel, the pharmaceutically acceptable salt thereof, or the prodrug thereof is administered at a dosage of about 0.16±0.15 mg/kg, about 0.16±0.14 mg/kg, about 0.16±0.13 mg/kg, about 0.16±0.12 mg/kg, about 0.16±0.11 mg/kg, about 0.16±0.10 mg/kg, about 0.16±0.09 mg/kg, about 0.16±0.08 mg/kg, about 0.16±0.07 mg/kg, about 0.16±0.06 mg/kg, about 0.16±0.05 mg/kg, about 0.16±0.04 mg/kg, about 0.16±0.03 mg/kg, about 0.16±0.02 mg/kg, or about 0.16±0.01 mg/kg.

Exemplary Embodiment No. 45. The method, combination, tezampanel, benzodiazepine, or use of any one of the previous Exemplary Embodiments, wherein the tezampanel, the pharmaceutically acceptable salt thereof, or the prodrug thereof is administered at a dosage of about 110 mg or lower, about 105 mg or lower, about 100 mg or lower, about 95 mg or lower, about 90 mg or lower, about 85 mg or lower, about 80 mg or lower, about 75 mg or lower, about 70 mg or lower, about 65 mg or lower, about 60 mg or lower, about 55 mg or lower, about 50 mg or lower, about 45 mg or lower, about 40 mg or lower, about 35 mg or lower, about 30 mg or lower, about 25 mg or lower, about 20 mg or lower, about 15 mg or lower, about 10 mg or lower, about 9 mg or lower, about 8 mg or lower, about 7 mg or lower, about 6 mg or lower, about 5 mg or lower, about 4 mg or lower, about 3 mg or lower, about 2 mg or lower, or about 1 mg or lower.

Exemplary Embodiment No. 46. The method, combination, tezampanel, benzodiazepine, or use of any one of the previous Exemplary Embodiments, wherein the tezampanel, the pharmaceutically acceptable salt thereof, or the prodrug thereof is administered at a dosage of about 1 mg or higher, about 2 mg or higher, about 3 mg or higher, about 4 mg or higher, about 5 mg or higher, about 6 mg or higher, about 7 mg or higher, about 8 mg or higher, about 9 mg or higher, about 10 mg or higher, about 15 mg or higher, about 20 mg or higher, about 25 mg or higher, about 30 mg or higher, about 35 mg or higher, about 40 mg or higher, about 45 mg or higher, about 50 mg or higher, about 55 mg or higher, about 60 mg or higher, about 65 mg or higher, about 70 mg or higher, about 75 mg or higher, about 80 mg or higher, about 85 mg or higher, about 90 mg or higher, about 95 mg or higher, or about 100 mg or higher.

Exemplary Embodiment No. 47. The method, combination, tezampanel, benzodiazepine, or use of any one of the previous Exemplary Embodiments, wherein the therapeutically effective amount of tezampanel, the pharmaceutically acceptable salt thereof, or the prodrug thereof is administered in a parenteral dosage form.

Exemplary Embodiment No. 48. The method, combination, tezampanel, benzodiazepine, or use of any one of the previous Exemplary Embodiments, wherein the parenteral dosage form is an intravenous, intramuscular, subcutaneous, or intradermal dosage form.

Exemplary Embodiment No. 49. The method, combination, tezampanel, benzodiazepine, or use of any one of the previous Exemplary Embodiments, wherein the tezampanel, the pharmaceutically acceptable salt thereof, or the prodrug thereof is administered at a dosage about 0.8 mg/kg or lower, about 0.7 mg/kg or lower, about 0.6 mg/kg or lower, about 0.5 mg/kg or lower, about 0.4 mg/kg or lower, about 0.3 mg/kg or lower, about 0.2 mg/kg or lower, about 0.1 mg/kg or lower, about 0.09 mg/kg or lower, about 0.08 mg/kg or lower, about 0.07 mg/kg or lower, about 0.06 mg/kg or lower, about 0.05 mg/kg or lower, about 0.04 mg/kg or lower, about 0.03 mg/kg or lower, about 0.02 mg/kg or lower, or about 0.01 mg/kg or lower.

Exemplary Embodiment No. 50. The method, combination, tezampanel, benzodiazepine, or use of any one of the previous Exemplary Embodiments, wherein the tezampanel, the pharmaceutically acceptable salt thereof, or the prodrug thereof is administered at a dosage of about 0.01 mg/kg or higher, about 0.02 mg/kg or higher, about 0.03 mg/kg or higher, about 0.04 mg/kg or higher, about 0.05 mg/kg or higher, about 0.06 mg/kg or higher, about 0.07 mg/kg or higher, about 0.08 mg/kg or higher, about 0.09 mg/kg or higher, about 0.1 mg/kg or higher, about 0.2 mg/kg or higher, about 0.3 mg/kg or higher, about 0.4 mg/kg or higher, about 0.5 mg/kg or higher, about 0.6 mg/kg or higher, or about 0.7 mg/kg or higher.

Exemplary Embodiment No. 51. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated one or more times during the treatment.

Exemplary Embodiment No. 52. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated once, twice, three times, four times, five times, six times, seven times, or eight times during the treatment.

Exemplary Embodiment No. 53. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated once during the treatment.

Exemplary Embodiment No. 54. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated twice during the treatment.

Exemplary Embodiment No. 55. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated three times during the treatment.

Exemplary Embodiment No. 56. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated every one hour, every two hours, every three hours, every four hours, every five hours, every six hours, every seven hours, every eight hours, every nine hours, every ten hours, every 11 hours, every 12 hours, every 13 hours, every 14 hours, every 15 hours, every 16 hours, every 17 hours, every 18 hours, every 19 hours, every 20 hours, every 21 hours, every 22 hours, every 23 hours, or every 24 hours.

Exemplary Embodiment No. 57. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated every four hours.

Exemplary Embodiment No. 58. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated every six hours.

Exemplary Embodiment No. 59. The method, combination, tezampanel, benzodiazepine, or use of any one of the previous Exemplary Embodiments, wherein the administration of the therapeutically effective amount of tezampanel, the pharmaceutically acceptable salt thereof, or the prodrug thereof is repeated every eight hours.

Exemplary Embodiment No. 60. The method, combination, tezampanel, benzodiazepine, or use of any one of the previous Exemplary Embodiments, wherein the benzodiazepine is clorazepate, diazepam, flurazepam, halazepam, prazepam, lorazepam, lormetazepam, oxazepam, temazepam, clonazepam, flunitrazepam, nimetazepam, nitrazepam, adinazolam, alprazolam, estazolam, triazolam, climazolam, loprazolam, or midazolam.

Exemplary Embodiment No. 61. The method, combination, tezampanel, benzodiazepine, or use of any one of the previous Exemplary Embodiments, wherein the benzodiazepine is clorazepate, diazepam, flurazepam, halazepam, or prazepam.

Exemplary Embodiment No. 62. The method, combination, tezampanel, benzodiazepine, or use of any one of the previous Exemplary Embodiments, wherein the benzodiazepine is lorazepam, lormetazepam, oxazepam, or temazepam.

Exemplary Embodiment No. 63. The method, combination, tezampanel, benzodiazepine, or use of any one of the previous Exemplary Embodiments, wherein the benzodiazepine is clonazepam, flunitrazepam, nimetazepam, or nitrazepam.

Exemplary Embodiment No. 64. The method, combination, tezampanel, benzodiazepine, or use of any one of the previous Exemplary Embodiments, wherein the benzodiazepine is adinazolam, alprazolam, estazolam, or triazolam.

Exemplary Embodiment No. 65. The method, combination, tezampanel, benzodiazepine, or use of any one of the previous Exemplary Embodiments, wherein the benzodiazepine is climazolam, loprazolam, or midazolam.

Exemplary Embodiment No. 66. The method, combination, tezampanel, benzodiazepine, or use of any one of the previous Exemplary Embodiments, wherein the therapeutically effective amount of the benzodiazepine, the pharmaceutically acceptable salt thereof, or the prodrug thereof reduces the frequency, severity, or duration of the seizure.

Exemplary Embodiment No. 67. The method, combination, tezampanel, benzodiazepine, or use of any one of the previous Exemplary Embodiments, wherein the therapeutically effective amount of the benzodiazepine, the pharmaceutically acceptable salt thereof, or the prodrug thereof arrests the seizure.

Exemplary Embodiment No. 68. The method, combination, tezampanel, benzodiazepine, or use of any one of the previous Exemplary Embodiments, wherein the therapeutically effective amount of the benzodiazepine, the pharmaceutically acceptable salt thereof, or the prodrug thereof alleviates an adversing effect of the benzodiazepine, the pharmaceutically acceptable salt thereof, or the prodrug thereof.

Exemplary Embodiment No. 69. The method, combination, tezampanel, benzodiazepine, or use of any one of the previous Exemplary Embodiments, wherein the adversing effect is a sedating effect.

Exemplary Embodiment No. 70. The method, combination, tezampanel, benzodiazepine, or use of any one of the previous Exemplary Embodiments, wherein the therapeutically effective amount of the benzodiazepine, the pharmaceutically acceptable salt thereof, or the prodrug thereof is administered in an oral dosage form Exemplary Embodiment No. 71. The method, combination, tezampanel, benzodiazepine, or use of any one of the previous Exemplary Embodiments, wherein the oral dosage form is a tablet.

Exemplary Embodiment No. 72. The method, combination, tezampanel, benzodiazepine, or use of any one of the previous Exemplary Embodiments, wherein the therapeutically effective amount of the benzodiazepine, the pharmaceutically acceptable salt thereof, or the prodrug thereof is administered in a parenteral dosage form.

Exemplary Embodiment No. 73. The method, combination, tezampanel, benzodiazepine, or use of any one of the previous Exemplary Embodiments, wherein the parenteral dosage form is an intravenous, intramuscular, subcutaneous, or intradermal dosage form.

Exemplary Embodiment No. 74. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated one or more times during the treatment.

Exemplary Embodiment No. 75. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated once, twice, three times, four times, five times, six times, seven times, or eight times during the treatment.

Exemplary Embodiment No. 76. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated once during the treatment.

Exemplary Embodiment No. 77. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated twice during the treatment.

Exemplary Embodiment No. 78. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated three times during the treatment.

Exemplary Embodiment No. 79. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated every one hour, every two hours, every three hours, every four hours, every five hours, every six hours, every seven hours, every eight hours, every nine hours, every ten hours, every 11 hours, every 12 hours, every 13 hours, every 14 hours, every 15 hours, every 16 hours, every 17 hours, every 18 hours, every 19 hours, every 20 hours, every 21 hours, every 22 hours, every 23 hours, or every 24 hours.

Exemplary Embodiment No. 80. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated every four hours.

Exemplary Embodiment No. 81. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated every six hours.

Exemplary Embodiment No. 82. The method, combination, tezampanel, benzodiazepine, or use of any one of the previous Exemplary Embodiments, wherein the administration of the therapeutically effective amount of the benzodiazepine, the pharmaceutically acceptable salt thereof, or the prodrug thereof is repeated every eight hours.

Exemplary Embodiment No. 83. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered simultaneously.

Exemplary Embodiment No. 84. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered sequentially.

Exemplary Embodiment No. 85. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered alternatively.

Exemplary Embodiment No. 86. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in temporal proximity.

Exemplary Embodiment No. 87. The method, combination, tezampanel, benzodiazepine, or use of any one of the previous Exemplary Embodiments, wherein the therapeutically effective amount of tezampanel, the pharmaceutically acceptable salt thereof, or the prodrug thereof is administered within about 1 minutes, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 18 hours, or about 24 hours after the administration of the therapeutically effective amount of the benzodiazepine, the pharmaceutically acceptable salt thereof.

Exemplary Embodiment No. 88. The method, combination, tezampanel, benzodiazepine, or use of any one of the previous Exemplary Embodiments, wherein the therapeutically effective amount of the benzodiazepine, the pharmaceutically acceptable salt thereof, or the prodrug thereof is administered within about 1 minutes, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 18 hours, or about 24 hours after the administration of the therapeutically effective amount of tezampanel, the pharmaceutically acceptable salt thereof.

Exemplary Embodiment No. 89. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in different administration routes.

Exemplary Embodiment No. 90. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in a same administration route (e.g., oral administration).

Exemplary Embodiment No. 91. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in separate formulations.

Exemplary Embodiment No. 92. The method, combination, tezampanel, benzodiazepine, or use of any one of the previous Exemplary Embodiments, wherein (i) the therapeutically effective amount of tezampanel, the pharmaceutically acceptable salt thereof, or the prodrug thereof and (ii) the therapeutically effective amount of the benzodiazepine, the pharmaceutically acceptable salt thereof, or the prodrug thereof are administered in a co-formulation.

Exemplary Embodiment No. 93. The method, combination, tezampanel, benzodiazepine, or use of any one of the previous Exemplary Embodiments, wherein the method further comprises administering to the subject a supportive care.

Exemplary Embodiment No. 94. The method, combination, tezampanel, benzodiazepine, or use of any one of the previous Exemplary Embodiments, wherein the supportive care comprises an anticholinergic agent. The method, combination, tezampanel, benzodiazepine, or use of any one of the previous Exemplary Embodiments, wherein the supportive care comprises atropine.

Exemplary Embodiment No. 95. The method, combination, tezampanel, benzodiazepine, or use of any one of the previous Exemplary Embodiments, wherein the supportive care comprises an oxime being capable of protecting the active site of acetylcholinesterase.

Exemplary Embodiment No. 96. The method, combination, tezampanel, benzodiazepine, or use of any one of the previous Exemplary Embodiments, wherein the supportive care comprises pralidoxime.

Exemplary Embodiment No. 97. The method, combination, tezampanel, benzodiazepine, or use of any one of the previous Exemplary Embodiments, wherein the supportive care comprises an anticonvulsant.

Exemplary Embodiment No. 98. The method, combination, tezampanel, benzodiazepine, or use of any one of the previous Exemplary Embodiments, wherein the supportive care comprises diazepam.

Exemplary Embodiment No. 99. The method, combination, tezampanel, benzodiazepine, or use of any one of the previous Exemplary Embodiments, wherein the supportive care comprises oxygen supplementation.

Exemplary Embodiment No. 100. The method, combination, tezampanel, benzodiazepine, or use of any one of the previous Exemplary Embodiments, wherein the supportive care comprises warmth, hydration, or a combination thereof.

Exemplary Embodiment No. 101. The method, combination, tezampanel, benzodiazepine, or use of any one of the previous Exemplary Embodiments, wherein the supportive care comprises warmth.

Exemplary Embodiment No. 102. The method, combination, tezampanel, benzodiazepine, or use of any one of the previous Exemplary Embodiments, wherein the supportive care comprises a heating source.

Exemplary Embodiment No. 103. The method, combination, tezampanel, benzodiazepine, or use of any one of the previous Exemplary Embodiments, wherein the supportive care comprises a blanket.

Exemplary Embodiment No. 104. The method, combination, tezampanel, benzodiazepine, or use of any one of the previous Exemplary Embodiments, wherein the supportive care comprises hydration.

Exemplary Embodiment No. 105. The method, combination, tezampanel, benzodiazepine, or use of any one of the previous Exemplary Embodiments, wherein the supportive care comprises an intravenous fluid.

Exemplary Embodiment No. 106. The method, combination, tezampanel, benzodiazepine, or use of any one of the previous Exemplary Embodiments, wherein the administration reduces the frequency, severity, or duration of the seizure.

Exemplary Embodiment No. 107. The method, combination, tezampanel, benzodiazepine, or use of any one of the previous Exemplary Embodiments, wherein the therapeutically effective amount of tezampanel, the pharmaceutically acceptable salt thereof, or the prodrug thereof arrests the seizure.

Exemplary Embodiment No. 108. The method, combination, tezampanel, benzodiazepine, or use of any one of the previous Exemplary Embodiments, wherein the frequency, severity, and/or duration of the seizure in the subject is measured by a Likert Scale Score (LSS).

Exemplary Embodiment No. 109. The method, combination, tezampanel, benzodiazepine, or use of any one of the previous Exemplary Embodiments, wherein the administration results in a reduced LSS of the subject.

Exemplary Embodiment No. 110. The method, combination, tezampanel, benzodiazepine, or use of any one of the previous Exemplary Embodiments, wherein the subject has a LLS of about 3 or lower, about 2 or lower, or about 1 or lower, as compared to a comparable subject without administration of tezampanel, the pharmaceutically acceptable salt thereof, or the prodrug thereof, at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, or about 48 hours after the administration of tezampanel, the pharmaceutically acceptable salt thereof, or the prodrug thereof.

Exemplary Embodiment No. 111. The method, combination, tezampanel, benzodiazepine, or use of any one of the previous Exemplary Embodiments, wherein the subject has a LLS of about 6 or lower, about 5 or lower, about 4 or lower, about 3 or lower, about 2 or lower, or about 1 or lower, at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, or about 48 hours after the administration of tezampanel, the pharmaceutically acceptable salt thereof, or the prodrug thereof.

Exemplary Embodiment No. 112. The method, combination, tezampanel, benzodiazepine, or use of any one of the previous Exemplary Embodiments, wherein the subject has a LLS of about 5 or lower, about 4 or lower, about 3 or lower, about 2 or lower, or about 1 or lower, at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 30 hours, about 36 hours, about 42 hours, or about 48 hours after the administration of tezampanel, the pharmaceutically acceptable salt thereof, or the prodrug thereof.

Exemplary Embodiment No. 113. The method, combination, tezampanel, benzodiazepine, or use of any one of the previous Exemplary Embodiments, wherein the subject has a LLS of about 4 or lower, about 3 or lower, about 2 or lower, or about 1 or lower, at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, or about 18 hours after the administration of tezampanel, the pharmaceutically acceptable salt thereof, or the prodrug thereof.

Exemplary Embodiment No. 114. The method, combination, tezampanel, benzodiazepine, or use of any one of the previous Exemplary Embodiments, wherein the subject has a LLS of about 3 or lower, about 2 or lower, or about 1 or lower, at about 10 minutes, about 20 minutes, about 30 minutes, about 40 minutes, about 50 minutes, about 1 hour, about 1.5 hours, about 2 hours, about 3 hours, about 4 hours, about 5 hours, about 6 hours, about 7 hours, about 8 hours, about 9 hours, about 10 hours, about 11 hours, or about 12 hours after the administration of tezampanel, the pharmaceutically acceptable salt thereof, or the prodrug thereof.

### Definitions

The term “about”, as used herein, generally means±10% of the value stated. In some embodiments, “about” or “approximately” generally means±9%, ±8%, ±7%, ±6%, ±5%, ±4%, ±3%, ±2%, or ±1% of the stated value.

It is understood that tezampanel may be identified with the IUPAC name of (3S,4aR,6R,8aR)-6-[2-(1H-tetrazol-5-yl)ethyl]decahydroisoquinoline-3-carboxylic acid, the CAS No. 150131-78-5, and/or the following chemical structure:

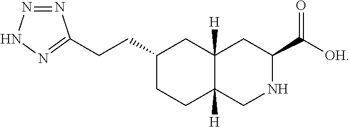 [SMILES: O=C(O)[C@@H]1C[C@H]2C[C@@H](CCc3nn[nH]n3)CC[C@H]2CN1]

In some embodiments, a prodrug of tezampanel may be administered. In some embodiments, the prodrug of tezampanel is dasolampanel. It is understood that dasolampanel may be identified with the IUPAC name of (3S,4aS,6S,8aR)-6-(3-chloro-2-(1H-tetrazol-5-yl)phenoxy) decahydroisoquinoline-3-carboxylic acid, the CAS No. 503294-13-1, and/or the following chemical structure:

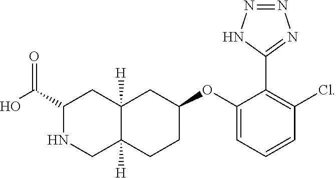

Compounds of the present disclosure that contain nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺—O⁻). Furthermore, in other instances, the nitrogens in the compounds of the present disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

In the present specification, the structural formula of the compound represents a certain isomer for convenience in some cases, but the present disclosure includes all isomers, such as optical isomers based on an asymmetrical carbon, stereoisomers, tautomers, and the like, it being understood that not all isomers may have the same level of activity. In addition, a crystal polymorphism may be present for the compounds represented by the formula. It is noted that any crystal form, crystal form mixture, or anhydride or hydrate thereof is included in the scope of the present disclosure.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

It is to be understood that the compounds of any Formula described herein include the compounds themselves, as well as their salts, and their solvates, if applicable. A salt, for example, can be formed between an anion and a positively charged group (e.g., amino) on a substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted benzene compound.

Suitable cations include sodium ion, potassium ion, magnesium ion, calcium ion, and an ammonium cation such as tetramethylammonium ion. The substituted benzene compounds also include those salts containing quaternary nitrogen atoms.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure, and are substituted with various groups as described herein.

As used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

One of ordinary skill in the art will note that, during the reaction sequences and synthetic schemes described herein, the order of certain steps may be changed, such as the introduction and removal of protecting groups. One of ordinary skill in the art will recognize that certain groups may require protection from the reaction conditions via the use of protecting groups. Protecting groups may also be used to differentiate similar functional groups in molecules. A list of protecting groups and how to introduce and remove these groups can be found in Greene, T. W., Wuts, P. G. M., Protective Groups in Organic Synthesis, 3^{rd} edition, John Wiley & Sons: New York, 1999.

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

As used herein, the term “temporal proximity” refers to that administration of one therapeutic agent occurs within a time period before or after the administration of another therapeutic agent, such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within about 1 minutes, about 2 minutes, about 3 minutes, about 4 minutes, about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 1 hour, about 2 hours, about 3 hours, about 4 hours, about 5 hours, about 6 hours, about 7 hours, about 8 hours, about 9 hours, about 10 hours, about 12 hours, about 18 hours, or about 24 hours. In some embodiments, multiple administration of one therapeutic agent can occur in temporal proximity to a single administration of another therapeutic agent. In some embodiments, temporal proximity may change during a treatment cycle or within a dosing regimen.

As used herein, the term “prodrug” refers to any agent which, when administered to a mammal, is converted in whole or in part to a targeted compound. In some embodiments, the prodrug of a compound is also a pharmaceutically acceptable salt of the compound.

It is to be understood that a compound of the present disclosure, or a pharmaceutically acceptable salt, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{rd} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990), Mandell, et al., Principles and Practice of Infectious Diseases, Saunders Publishing (8th edition, 2014). These texts can, of course, also be referred to in making or using an aspect of the disclosure.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease and also preferably causing complete regression of the disease. Dosages can range from about 0.01 mg/kg per day to about 5000 mg/kg per day. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. In some embodiments, the pharmaceutically acceptable salt of a compound is also a prodrug of the compound. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is to be understood that the compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice ofPharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### Examples

### Example 1. Effect of Tezampanel in Arresting Neve Agent Induced Seizures in Rodents

EEG Implant Surgery: The rat is covered with sterilized drape, fenestrated to expose the dorsal cranial surgical area. A central midline incision is made near the occipital bone, extending rostral towards between the orbits, and underlying tissue dissected in order to expose the dorsal cranial suture junctions (i.e. lambda and bregma). 5 holes are drilled through the skull, attempting to create a channel to, but not penetrating, the dura. Stainless steel screws are placed in the following locations: slightly behind the Bregma on each hemisphere, rostral to the lambda on each hemisphere, in addition to a referential screw positioned over the frontal area. The screws are attached to insulated stainless steel electrode wires. The screws and attached pedestal are cemented into place with dental cement (e.g. Dentalon Plus®), osteobond (e.g. Zimmer Palacos R+G®), or another similar material. The skin is closed with absorbable, monofilament sutures (e.g. 4-0 Monocryl).

Sarin, Atropine, Pralidoxime and Midazolam Administration: Following 1-week recovery from EEG implant surgery, animals receive 1xLD50s (160 μg/kg) of sarin by subcutaneous administration followed by atropine methyl nitrate (2.53 mg/kg, IM; this dose corresponds to 2 mg/kg of atropine base) and pralidoxime (25 mg/kg, IM) at 1 minute (±0.25 min). Seizure activity typically begins at 10-15 minutes following sarin administration. All animals receive a single injection of midazolam (0.66 mg/kg, IM) at 50 minutes post sarin administration.

Treatment Group Assignments: At 60 minutes post sarin exposure, animals were randomized to receive either normal saline (control group) by an IM route or a dose of tezampanel (2.5, 5, 7.5 or 15 mg/kg) by an IM route. In one cohort, the administration of tezampanel was delayed for 6 hours post sarin to determine if tezampanel would remain effective when given in a greatly delayed manner.

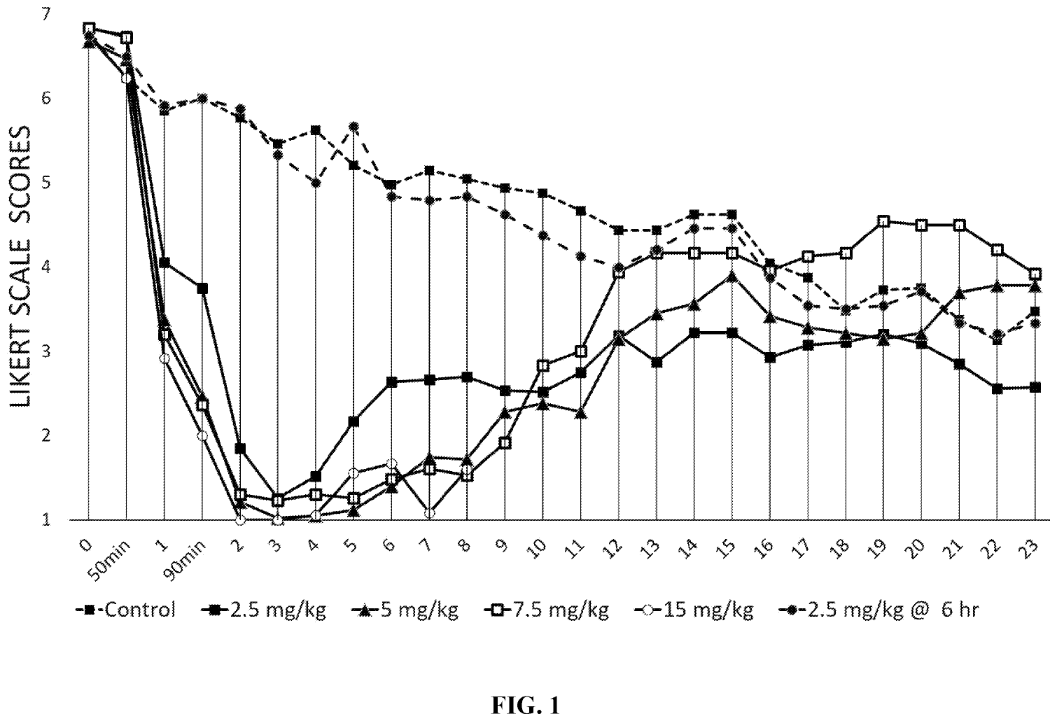

EEG Monitoring: In all animals, EEGs were monitored by a computerized data acquisition system to capture the initiation and subsequent attempts to arrest nerve agent induced seizure. EEG data was analyzed by a 7-point Likert Scale and condensed into hourly averages over a 24 hour period (see FIG. 1).

### EQUIVALENTS

It is to be understood that the invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method of treating a disease in a subject in need thereof, comprising administering to the subject:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

2. A method of treating a disease resistant to a treatment with the benzodiazepine in a subject in need thereof, comprising administering to the subject a therapeutically effective amount of tezampanel, a pharmaceutically acceptable salt thereof, or a prodrug thereof.

3. A method of treating a disease resistant to a treatment with the tezampanel in a subject in need thereof, comprising administering to the subject a therapeutically effective amount of benzodiazepine, a pharmaceutically acceptable salt thereof, or a prodrug thereof.

4. A combination for treating a disease in a subject in need thereof, wherein the combination comprises:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

5. Tezampanel, a pharmaceutically acceptable salt, or a prodrug thereof for use in treating a disease resistant to a treatment with benzodiazepine in a subject in need thereof.

6. Benzodiazepine, a pharmaceutically acceptable salt, or a prodrug thereof for use in treating a disease resistant to a treatment with tezampanel in a subject in need thereof.

7. Use of a combination in the manufacture of a medicament for treating a disease in a subject in need thereof, wherein the combination comprises:
(i) a therapeutically effective amount of tezampanel, a pharmaceutically acceptable salt thereof, or a prodrug thereof; and
(ii) a therapeutically effective amount of a benzodiazepine, a pharmaceutically acceptable salt thereof, or a prodrug thereof.

8. Use of tezampanel, a pharmaceutically acceptable salt, or a prodrug thereof in the manufacture of a medicament for treating a disease resistant to a treatment with benzodiazepine in a subject in need thereof.

9. Use of benzodiazepine, a pharmaceutically acceptable salt, or a prodrug thereof in the manufacture of a medicament for treating a disease resistant to a treatment with tezampanel in a subject in need thereof.

10. The method, combination, tezampanel, benzodiazepine, or use of any one of the previous claims, wherein the subject is a human.

11. The method, combination, tezampanel, benzodiazepine, or use of any one of the previous claims, wherein the disease is a seizure.

12. The method, combination, tezampanel, benzodiazepine, or use of any one of the previous claims, wherein the seizure is an agent-induced seizure.

13. The method, combination, tezampanel, benzodiazepine, or use of any one of the previous claims, wherein the inducing agent is a nerve agent.

14. The method, combination, tezampanel, benzodiazepine, or use of any one of the previous claims, wherein the seizure is resistant to a treatment without tezampanel, the pharmaceutically acceptable salt thereof, or the prodrug thereof.

15. The method, combination, tezampanel, benzodiazepine, or use of any one of the previous claims, wherein the seizure is resistant to a treatment with the benzodiazepine, the pharmaceutically acceptable salt thereof, or the prodrug thereof and without tezampanel, the pharmaceutically acceptable salt thereof, or the prodrug thereof.

16. The method, combination, tezampanel, benzodiazepine, or use of any one of the previous claims, wherein the therapeutically effective amount of tezampanel, the pharmaceutically acceptable salt thereof, or the prodrug thereof reduces the frequency, severity, or duration of the seizure.

17. The method, combination, tezampanel, benzodiazepine, or use of any one of the previous claims, wherein the therapeutically effective amount of tezampanel, the pharmaceutically acceptable salt thereof, or the prodrug thereof arrests the seizure.

18. The method, combination, tezampanel, benzodiazepine, or use of any one of the previous claims, wherein the therapeutically effective amount of tezampanel, the pharmaceutically acceptable salt thereof, or the prodrug thereof alleviates an adversing effect of tezampanel, the pharmaceutically acceptable salt thereof, or the prodrug thereof.

19. The method, combination, tezampanel, benzodiazepine, or use of any one of the previous claims, wherein the benzodiazepine is clorazepate, diazepam, flurazepam, halazepam, prazepam, lorazepam, lormetazepam, oxazepam, temazepam, clonazepam, flunitrazepam, nimetazepam, nitrazepam, adinazolam, alprazolam, estazolam, triazolam, climazolam, loprazolam, or midazolam.

20. The method, combination, tezampanel, benzodiazepine, or use of any one of the previous claims, wherein the therapeutically effective amount of the benzodiazepine, the pharmaceutically acceptable salt thereof, or the prodrug thereof reduces the frequency, severity, or duration of the seizure.

21. The method, combination, tezampanel, benzodiazepine, or use of any one of the previous claims, wherein the therapeutically effective amount of the benzodiazepine, the pharmaceutically acceptable salt thereof, or the prodrug thereof arrests the seizure.

22. The method, combination, tezampanel, benzodiazepine, or use of any one of the previous claims, wherein the therapeutically effective amount of the benzodiazepine, the pharmaceutically acceptable salt thereof, or the prodrug thereof alleviates an adversing effect of the benzodiazepine, the pharmaceutically acceptable salt thereof, or the prodrug thereof.

23. The method, combination, tezampanel, benzodiazepine, or use of any one of the previous claims, wherein the adversing effect is a sedating effect.

24. The method, combination, tezampanel, benzodiazepine, or use of any one of the previous claims, wherein the method further comprises administering to the subject a supportive care.

25. The method, combination, tezampanel, benzodiazepine, or use of any one of the previous claims, wherein the administration reduces the frequency, severity, or duration of the seizure.

26. The method, combination, tezampanel, benzodiazepine, or use of any one of the previous claims, wherein the frequency, severity, and/or duration of the seizure in the subject is measured by a Likert Scale Score (LSS).

27. The method, combination, tezampanel, benzodiazepine, or use of any one of the previous claims, wherein the administration results in a reduced LSS of the subject.
